(19) United States
(12) Patent Application Publication
(10) Pub. No.: US 2006/0052864 A1
(43) Pub. Date: Mar. 9, 2006

(54) Endoprosthesis comprising a magnesium alloy

(75) Inventors: Claus Harder, Uttenreuth (DE); Marc Kuttler, Berlin (DE); Bodo Gerold, Zellingen (DE); Heinz Mueller, Erlangen (DE)

Correspondence Address: HAHN LOESER & PARKS, LLP, One GOJO Plaza, Suite 300, AKRON, OH, 44311-1076, US

(73) Assignee: BIOTRONIK VI Patent AG, Baar (CH)

(21) Appl. No.: 11/221,344

(22) Filed: Sep. 7, 2005

(30) Foreign Application Priority Data
Sep. 7, 2004 (DE) 102004 043 232.5

(51) Int. Cl. A61F 2/06

(52) U.S. Cl. 623/1.38

(57) ABSTRACT

An endoprosthesis, in particular an intraluminal endoprosthesis such as a stent, comprises a carrier structure, which includes at least one component comprising a magnesium alloy of the following composition: Rare earth metals: between about 2.0 and about 5.0% by weight, with neodymium between about 1.5 and about 3.0% by weight Yttrium: between about 3.5% and about 4.5% by weight Zirconium: between about 0.3% and about 1.0% by weight Balance: between 0 and about 0.5% by weight wherein magnesium occupies the proportion by weight that remains to 100% by weight in the alloy.

### BACKGROUND OF THE INVENTION

The invention concerns an endoprosthesis, in particular an intraluminal endoprosthesis such as a stent, having a carrier structure which entirely or in parts comprises a magnesium alloy.

The purpose of many endoprostheses is to implement a support function in the interior of the body of a patient. Accordingly, endoprostheses are designed to be implantable and have a carrier structure which ensures the support function. Implants of metallic materials are known. The choice of metals as the material for the carrier structure of an implant of that nature is based in particular on the mechanical properties of metals.

Metallic stents are known in large numbers. One of the main areas of use of such stents is permanently dilating and holding open vessel constrictions, in particular constrictions (stenoses) of the coronary vessels. In addition, aneurysm stents are also known, which afford a support function for a damaged vessel wall. Stents of that kind generally have a peripheral wall of sufficient carrying strength to hold the constricted vehicle open to the desired amount. In order to permit an unimpeded flow of blood through the stent, it is open at both ends. More complicated configurations also permit an unimpeded flow of blood in side vessels (side branch access). The supporting peripheral wall is generally formed by a lattice-like carrier structure which makes it possible for the stent to be introduced in a compressed condition, when it is of small outside diameter, to the constriction to be treated in the respective vessel and there expanded, for example, by means of a balloon catheter to such a degree that the vessel is of the desired enlarged inside diameter. Basically therefore, the stent is subject to the requirement that its carrier structure in the expanded condition affords a sufficient carrying strength to hold the vessel open. In order to avoid unnecessary vessel damage, it is also desirable that, after expansion and after removal of the balloon, the stent only slightly elastically springs back (recoil) so that upon expansion the stent only has to be expanded little beyond the desired final diameter. Further criteria which are desirable in relation to a stent include, for example, uniform surface coverage and a structure which allows a certain degree of flexibility in relation to the longitudinal axis of the stent.

In some cases, particularly in the case of such intraluminal endoprostheses as stents, a durable support function afforded by the endoprosthesis is not required. Rather, in some of those situations of use, the body tissue can recover in the presence of the support prosthesis in such a way that there is no need for an ongoing supporting action by the prosthesis. That has led to the idea of making such prostheses from bioresorbable material.

Besides the desired mechanical properties of a stent, as far as possible it should interact with the body tissue at the implantation location in such a way that renewed vessel constrictions do not occur, in particular vessel constrictions caused by the stent itself. Re-stenosis (re-constriction of the vessel) should be avoided as much as possible. It is also desirable if the stent is, as far as possible, responsible for no, or only a very slight, inflammatory effect. In regard to a biodegradable metal stent, it is moreover desirable if the decomposition products of the metal stent as far as possible have no, or only very little, negative physiological effects and even positive physiological effects.

DE 197 31 021 discloses a bioresorbable metal stent, the material of which, as its main constituent, contains magnesium, iron or zinc. The mechanical properties, degradation behavior and biocompatibility mean that in particular magnesium alloys are to be preferred.

In DE 102 53 634, DE 101 28 100 or EP 1 395 297 the focus is on the use of such biodegradable magnesium alloys. The magnesium alloys proposed in the last two publications contain aluminum. In that case, the aluminum is required inter alia for the formation of cover layers which are intended to slow down diffusion of the magnesium and thus the degradation process. According to those publications, that is required in order to achieve sufficiently long mechanical stability for the endoprosthesis and to prevent/alleviate outgassing phenomena in the degradation process.

Aluminum however, is known for causing damage to health, particularly when it is in ionic form. Thus aluminum is known, inter alia, for causing damage to the central nervous system and triggering symptoms such as dementia, memory loss, loss of motivation or intense shaking. Aluminum is considered as a risk factor for Alzheimer's disease (Harold D Foster Ph D, Journal für Orthomolekulare Medizin 2/01).

Adverse effects in regard to biocompatibility in the immediate proximity of endoprostheses comprising Al-bearing magnesium alloys could also be observed in experiments. Thus, in animal experiments, pathological halos were observed around the degrading legs of such stents as well as pronounced neointima hyperplasia, which counteracts the real purpose of the stent of preventing vessel closure. The use of aluminum in endoprostheses, in particular stents, is thus undesirable.

Irritation of the surrounding tissue, which mostly involves inflammation and neointima hyperplasia and results in re-stenosis, is also triggered by the mechanical irritation of the implant, apart from lack of biocompatibility of the materials used (for example Al). Previous approaches for reducing mechanical irritation due to the implant are based on reducing the contact areas of the implant, which generate the irritation. Such methods however have limits, due to the required mechanical properties, as well as the blade effect which occurs as from a critical width.

Hitherto, the approach involving activating the healing processes of the body itself, in the context of using endoprostheses, in order in that way further to improve the healing process, has been generally neglected.

### BRIEF SUMMARY OF THE INVENTION

With that background in mind, an aspect of the present invention is to provide a biodegradable endoprosthesis based on a magnesium alloy, which avoids the outlined disadvantages of the state of the art. In particular, the invention aims to provide that degradation and biocompatibility properties are optimized and/or healing processes of the body itself are activated and promoted.

In accordance with the invention, that aspect attained by an endoprosthesis having a carrier structure which entirely or in parts comprises a magnesium alloy of the following composition:
Rare earth metals: between about 2.0 and about 5.0% by weight, with neodymium between about 1.5 and about 3.0% by weight
Yttrium: between about 3.5% and about 4.5% by weight
Zirconium: between about 0.3 and about 1.0% by weight, and
Balance: between 0 and about 0.5% by weight
   wherein magnesium occupies the proportion by weight in the alloy which remains to 100%. The alloy exhibits very advantageous mechanical and physiological properties and an advantageous degradation behavior in vivo. It can be easily processed and in initial studies exhibits a positive physiological effect on the surrounding tissue in a human and an animal if the alloy is used in endoprostheses, in particular, stents.

The collective term ‘rare earth metal’ stands for the elements scandium (atomic number 21), lanthanum (57) and the 14 elements following lanthanum: cerium (58), praseodymium (59), neodymium (60), promethium (61), samarium (62), europium (63), gadolinium (64), terbium (65), dysprosium (66), holmium (67), erbium (68), thulium (69), ytterbium (70) and lutetium (71), which are referred to as lanthanides. The proportion of the rare earth metals in the magnesium alloy thus also includes the proportion of neodymium. The latter proportion is also related to the total weight of the alloy and must be in the specified range. If the proportion of neodymium in the alloy is for example 2.0% by weight and the proportion of rare earth metals is 2.5% by weight, then necessarily rare earth metals, besides neodymium, have a proportion by weight in the alloy of 0.5% by weight.

The balance preferably contains only the impurities caused by the magnesium alloy production process. In other words, the composition preferably only contains specific impurities which cannot be avoided in production of the alloy or residual components which are deliberately added to the alloy. That ensures, and in part even first attains, the positive physiological effects and the mechanical properties of the material.

Supplemental to or alternatively to the above-indicated preferred variant, the balance contains no or at most <0.01% by weight of aluminum. It is precisely aluminum that has a pronounced adverse influence on physiological behavior as material investigations both in vivo and in vitro have shown.

By virtue of the adverse properties, in particular on biocompatibility, besides the element aluminum (Al), preferably also the elements copper (Cu), nickel (Ni), silver (Ag), mercury (Hg), cadmium (Cd), beryllium (Be) or chromium (Cr) are also avoided in the alloys; that is to say, the elements are not contained in the alloy, apart from impurities caused by the manufacturing procedure. The proportion in the alloy referred to as the balance contains as a matter of priority, proportions by mass of one, more or all of the stated elements, under the following limits:
Aluminum<0.01% by weight,
Copper<0.03% by weight,
Nickel<0.005% by weight.
Silver<0.01% by weight,
Mercury<0.03% by weight,
Cadmium<0.03% by weight,
Beryllium<0.03% by weight,
Chromium<0.03% by weight.

Avoiding those elements is of significance in terms of the purpose of the invention, as they have an effect which is damaging to health, they undesirably influence the mechanical properties of the alloy and they adversely affect the influences of the alloy and in particular, magnesium, which are positive influences in terms of the healing process. As is known, just slight traces of impurities can have a metallurgically and/or physiologically considerable effect. Identifying the troublesome elements and in particular, establishing limit values in respect of those elements therefore affords a considerable technical contribution to optimizing the products.

It is preferred, in contrast, for the balance to contain the elements lithium and/or zinc. The proportion of the components in the alloy is preferably between 0.1 and 0.5% by weight, wherein, in the case of adding lithium and zinc, the cumulated overall proportion thereof is at a maximum 0.5% by weight. The presence of those elements evidently positively influences the mechanical properties and biocompatibility of the implant.

The magnesium alloy described herein made it possible to achieve a significantly improved degradation process with markedly better reproducibility than is known hitherto, for example, for aluminum-bearing magnesium alloys (Heart (2003) 89, 691-656). In particular, reproducibility of the degradation process is indispensable for a medical use. By virtue of the controlled and slow degradation process embodied, no or at worst, slight, outgassing phenomena occur.

It was demonstrated in vivo and in vitro that the alloy and the decomposition products thereof are extremely biocompatible. By using the magnesium alloy, it was possible to counteract severe immunological reactions on the part of the body. Controlled cell growth, in particular in respect of human smooth muscle cells and endothelium cells, could be demonstrated on the basis of in vitro tests. Uncontrolled cell proliferation phenomena which can lead to re-stenosis appear to be prevented or greatly checked. That is not the case in that respect, in particular when using aluminum-bearing alloys in respect of which severe neointima hyperplasia was observed. The operative mechanism on which the positive effects are based has not hitherto been discovered in detail.

Magnesium could afford a contribution to the particular compatibility of the implant. Generally known effects and influences of magnesium, which is usually absorbed by way of food, on the body functions lead to the assumption that such processes are also at least locally activated when using magnesium as an implant in a suitable alloy composition.

It is known for example, that magnesium in an organism has a positive influence on wound healing, as that is necessary for anaerobic metabolism and promotes normal granulation of the connective tissue and rather prevents uncontrolled cell growth (Dr med Dr sc Nat PG Seeger, SANUM-Post No 13/1990, 14-16).

A further positive aspect when using magnesium, is that the non-specific defense by way of the properdin system is operative only when magnesium is present and phagocytosis of bacteria by leucocytes experiences a stimulus by the magnesium. Accordingly, magnesium provides, inter alia, for combating infections by promoting or activating the immune system of the body and reduces susceptibility to infections. Unwanted inflammation phenomena caused by infection, because of contamination which can occur in the context of using an endoprosthesis, and which in turn can be triggers for re-stenosis, are thus counteracted.

The alloy used here also has a positive action against mechanically induced re-stenosis. That is achieved on the one hand by the mechanical properties of the alloy used, which are distinguished by a favorable modulus of elasticity. In addition, the generally known muscle-relaxing action of magnesium (Ca antagonist) is used to reduce mechanical irritations. It is to be expected that the magnesium in the alloy or the magnesium-bearing decomposition products upon degradation promote relaxation of the muscle cells in the more immediate proximity. That is advantageous, in particular in relation to stent uses, as not only is the mechanical irritation reduced but also the vessel can be additionally held open by the locally relaxed muscle tissue.

It is further preferred that—independently of each other—the proportion of neodymium is between 2.0 and 2.5% by weight, the proportion of yttrium is between 3.8% by weight and 4.5% by weight and the proportion of the rare earth metals is between 2.8% by weight and 3.4% by weight. That makes it possible to still further increase physiological compatibility of the alloy and its decomposition products and optimize the degradation behavior for the intended purposes.

The magnesium alloy is preferably extruded. It has been found that processing of the alloy influences the physiological effect thereof. Those physiological properties are thus, at least in part, governed by the production process.

The endoprosthesis is preferably in the form of an intraluminal endoprosthesis. A particularly preferred endoprosthesis is one which is in the form of a stent, more particularly, a coronary stent or a peripheral stent. By virtue of the positive properties of the specified magnesium alloy, the carrier structure of the endoprosthesis preferably entirely consists of the magnesium alloy.

In accordance with a preferred variant for use of the alloy as a stent, in particular as a coronary stent or a peripheral stent, the specific composition of the magnesium alloy as well as the modification thereof is predetermined by the mode of manufacture and the stent design to the effect that decomposition starts immediately after implantation and mechanical integrity is maintained for between at least 5 days and at most 1 year. In that respect, the term ‘mechanical integrity’ is used to denote the stability, which is still sufficient in spite of progressing decomposition, of the structural elements of the implant, which serve to fulfil the medical purpose of the implant; that is to say, maintaining the required supporting function. In a particularly preferred feature, the period of time is between 10 and 90 days.

### BRIEF DESCRIPTION OF THE VIEWS OF THE DRAWINGS

The invention will now be described in greater detail by means of an embodiment with reference to the Figures in which:

FIG. 1 shows a diagrammatic view of an endoprosthesis in the form of a stent, and

FIG. 2 shows a development of the carrier structure of the stent shown in FIG. 1.

### DETAILED DESCRIPTION OF THE INVENTION

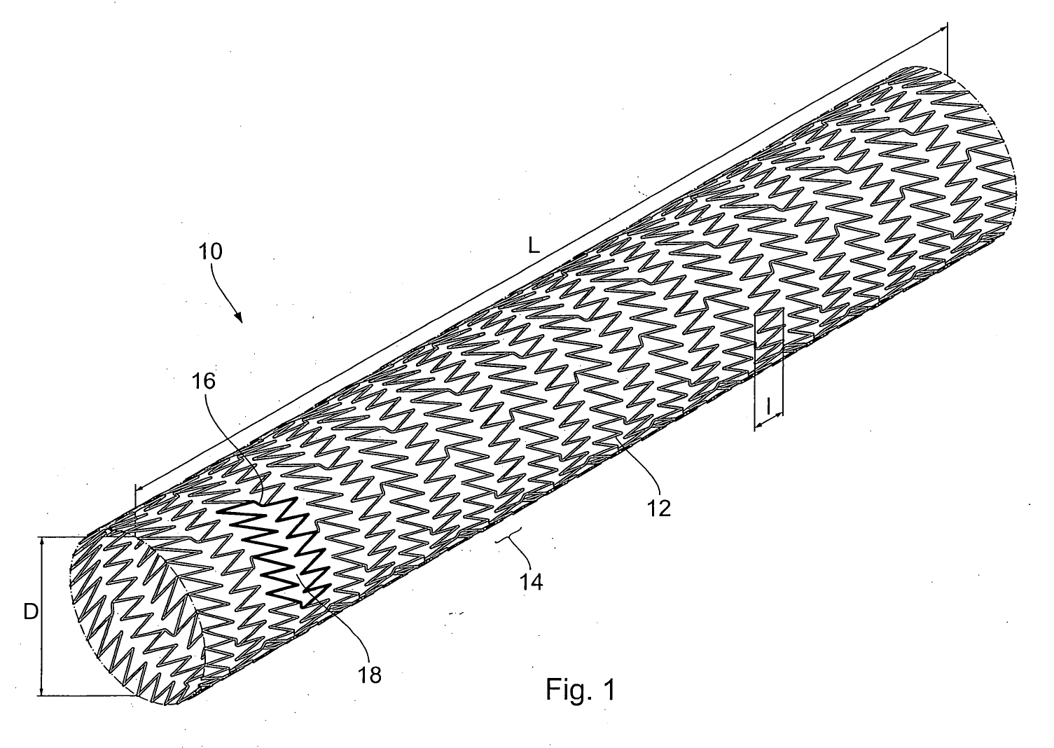

FIG. 1 shows an endoprosthesis as an endoluminal prosthesis in the form of a stent 10 having a carrier structure. The stent 10 and its carrier structure are in the form of a hollow body which is open at its ends and the peripheral wall of which is formed by the carrier structure which in turn is formed by partially folded legs 12. The legs 12 form support portions 14 which are each formed by a leg 12 which is closed in an annular configuration in the longitudinal direction and which is folded in a zig-zag or meander-shaped configuration. The stent is suitable for coronary use.

The carrier structure of the stent 10 is formed by a plurality of such support portions 12 which occur in succession in the longitudinal direction. The support portions or leg rings 14 are connected together by way of connecting legs 16. Each two connecting legs 16 which are mutually adjacent in the peripheral direction and the parts, which are in mutually opposite relationship between those connecting legs 16, of the leg rings 14 or support portions 12, define a mesh 18 of the stent 10. Such a mesh 18 is shown emphasized in FIG. 1. Each mesh 18 encloses a radial opening in the peripheral wall or the carrier structure of the stent 10.

Each leg ring 14 has between some three and six connecting legs 16 which are distributed equally over the periphery of the stent 10 and which respectively connect a leg ring 14 to the adjacent leg ring 14. Accordingly, the stent 10 has between three and six respective meshes 18 in the peripheral direction between two support portions 14.

The stent 10 is expandable in the peripheral direction by virtue of the folding of the legs 12. That is effected for example, by means of a per se known balloon catheter which at its distal end, has a balloon which is expandable by means of a fluid. The stent 10 is crimped onto the deflated balloon, in the compressed condition. Upon expansion of the balloon both the balloon and also the stent 10 are enlarged. The balloon can then be deflated again and the stent 10 is released from the balloon. In that way, the catheter can serve simultaneously for introducing the stent 10 into a blood vessel and in particular into a constricted coronary vessel and also for expanding the stent 10 at that location.

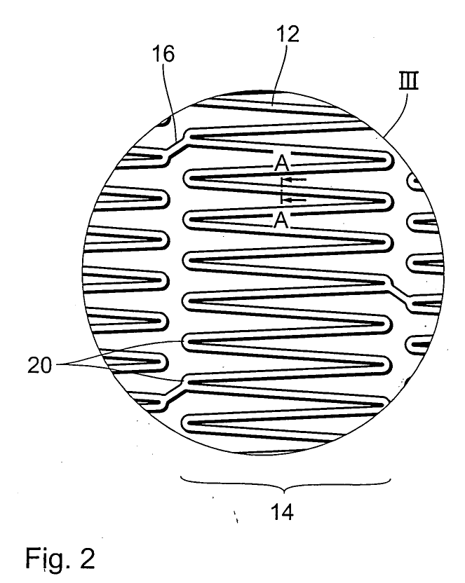

FIG. 2 shows a portion from a development of the peripheral wall of the stent 10. The development shows the compressed condition of the stent 10.

The carrier structure of the stent 10 shown in the Figures completely consists of a biodegradable magnesium alloy of the following composition:
Rare earth metals: 3.0% by weight, with neodymium 2.3% by weight
Yttrium: 4.0% by weight
Zirconium: 0.5% by weight
Lithium: 0.4% by weight
Aluminum, silver, copper, mercury, cadmium, beryllium, chromium and nickel: <0.005% by weight * (* detection limit in determination),
   wherein magnesium occupies the proportion by weight in the alloy which remains to 100%.

1. An endoprosthesis comprising a carrier structure which includes at least one component comprising a magnesium alloy of the following composition:
Rare earth metals: between about 2.0 and about 5.0% by weight, with neodymium between about 1.5 and about 3.0% by weight
yttrium: between about 3.5% and about 4.5% by weight
zirconium: between about 0.3% and about 1.0% by weight
balance: between 0 and about 0.5% by weight
wherein magnesium occupies the proportion by weight that remains to 100% by weight in the alloy:

2. An endoprosthesis as set forth in claim 1, wherein the proportion of yttrium is between 3.8% by weight and 4.5% by weight.

3. An endoprosthesis as set forth in claim 1, wherein the proportion of rare earth metals is between 2.8% by weight and 3.4% by weight.

4. An endoprosthesis as set forth in claim 1, wherein the proportion of neodymium is between 2.0% by weight and 2.5% by weight.

5. An endoprosthesis as set forth in claim 1, wherein the balance contains only the impurities governed by the magnesium alloy production process.

6. An endoprosthesis as set forth in claim 1, wherein the balance contains <0.01% by weight of aluminum.

7. An endoprosthesis as set forth in claim 1, wherein the balance contains <0.03% by weight of copper.

8. An endoprosthesis as set forth in claim 1, wherein the balance contains <0.005% by weight of nickel.

9. An endoprosthesis as set forth in claim 1, wherein the balance contains <0.01% by weight of silver.

10. An endoprosthesis as set forth in claim 1, wherein the balance contains <0.03% by weight of mercury.

11. An endoprosthesis as set forth in claim 1, wherein the balance contains <0.03% by weight of cadmium.

12. An endoprosthesis as set forth in claim 1, wherein the balance contains <0.03% by weight of chromium.

13. An endoprosthesis as set forth in claim 1, wherein the balance contains <0.03% by weight of beryllium.

14. An endoprosthesis as set forth in claim 1, wherein the balance contains lithium, zinc, or a combination thereof.

15. An endoprosthesis as set forth in claim 1, wherein the endoprosthesis is in the form of an intraluminal endoprosthesis.

16. An endoprosthesis as set forth in claim 15, wherein the endoprosthesis is in the form of a stent.

17. An endoprosthesis as set forth in claim 16, wherein the endoprosthesis is in the form of a coronary stent or a peripheral stent.

18. An endoprosthesis as set forth in claim 17, wherein a specific composition of the magnesium alloy and the modification thereof are predetermined to the effect that decomposition starts immediately after implantation and mechanical integrity is maintained for between at least 5 days and at most 1 year.

19. A method of making an endoprosthesis, the method comprising extruding a magnesium alloy of the following composition:
Rare earth metals: between about 2.0 and about 5.0% by weight, with neodymium between about 1.5 and about 3.0% by weight
Yttrium: between about 3.5% and about 4.5% by weight
Zirconium: between about 0.3% and about 1.0% by weight
Balance: between 0 and about 0.5% by weight
wherein magnesium occupies the proportion by weight that remains to 100% by weight in the alloy to form at least one component of the endoprosthesis.

20. The method of claim 19, wherein the endoprothesis is a stent.
